(19) United States
(12) Patent Application Publication
(10) Pub. No.: US 2025/0312369 A1
(43) Pub. Date: Oct. 9, 2025

(54) ANTI-AGING AND ANTI-INFLAMMATORY COMPOSITIONS COMPRISING NICOTINAMIDE MONONUCLEOTIDE AND RESVERATROL AND USES THEREOF

(71) Applicant: WONDERFEEL, San Francisco, CA (US)

(72) Inventors: Baran DILAVER, San Francisco, CA (US); Andrew Lurie SALZMAN, Herzliya (IL)

(21) Appl. No.: 18/865,265

(22) PCT Filed: May 10, 2023
(86) PCT No.: PCT/IB2023/054814

§ 371 Date: Nov. 12, 2024

Related U.S. Application Data
(60) Provisional application No. 63/341,034, filed on May 12, 2022.

(51) Int. Cl. A61K 31/706; A61K 31/05; A61K 31/4166; A61P 39/06

(52) U.S. Cl. CPC A61K 31/706; A61K 31/05; A61K 31/4166; A61P 39/06

(57) ABSTRACT

A composition containing nicotinamide mononucleotide (NMN) or an analog thereof, and certain antioxidants, more specifically resveratrol and at least one of ergothioneine and hydroxytyrosol, can be used in an anti-aging and/or anti-inflammatory method. The composition can contain NMN or nicotinamide riboside (NR) in an amount of from about 101 mg to about 1500 mg; the resveratrol in an amount of from about 50 mg to about 500 mg; ergothioneine or salt thereof, when present, in an amount of from about 3 mg to about 25 mg; and the hydroxytyrosol, when present, in an amount of from about 20 mg to about 100 mg.

### TECHNICAL FIELD

The present invention provides an anti-aging and anti-inflammatory composition comprising nicotinamide mononucleotide (NMN) or an analog thereof, and certain antioxidants, more specifically resveratrol and one of both of ergothioneine and hydroxytyrosol, and methods of use.

### BACKGROUND ART

Administration of NMN and its analogs has been shown in preclinical and clinical studies to be modestly efficacious in reducing inflammation and extending lifetime. The mechanism by which NMN and its analogs mediate these advantages are diverse, and include increased mitochondrial function, elevation in energetic intermediates such as nicotinamide adenine dinucleotide (NADH) and adenosine triphosphate (ATP), and higher levels of electron donors such as nicotinamide adenine dinucleotide phosphate (NADPH) which increase intracellular levels of antioxidant molecules such as reduced glutathione. Taken together, it is thought that the above alterations triggered by supplementation with NMN and its analogs reduce oxidant and inflammatory mediated stress and thereby improve cellular viability and function. The ability of NMN and its analogs, however, to effect these salutary effects is not sufficient to entirely relieve oxidant stress, especially in settings where the level of oxidant stress is severe.

### SUMMARY OF INVENTION

As has been postulated by the present inventors, in order to effectively relieve oxidant stress in settings where the level thereof is severe, it may be advantageous to provide additional antioxidant agents concomitant with administration of NMN and its analogs.

A wide range of antioxidants is known which might provide complementary benefit to NMN and its analogs. Among these are several natural products that are well established to be particularly safe and well tolerated. Combinations of antioxidants that differ in their chemical structure and thereby target different oxidant species and localize in different cellular domains are expected to act in an additive or synergistic function with NMN and its analogs. The basis of the present invention is thus the combination of NMN and its analogs with a diverse set of antioxidant molecules that taken together will provide a more comprehensive and effective antioxidant benefit.

Specifically, in one aspect, the present invention provides a composition, more particularly an anti-aging and anti-inflammatory composition, comprising nicotinamide mononucleotide (NMN, β-NMN) or an analog thereof such as nicotinamide adenine dinucleotide (NAD), nicotinamide riboside (NR), and niacinamide (NAM); resveratrol; and at least one of ergothioneine (L-ergothioneine) or a salt thereof, and hydroxytyrosol. Such a composition may be formulated as a pharmaceutical- or nutraceutical composition, optionally further comprising a pharmaceutically- or nutraceutically acceptable carrier, respectively.

The combination of antioxidants, anti-inflammatory agents, and mitochondrial energetic substrates disclosed herein may play an important role in the amelioration of diseases and conditions associated with inflammation and the natural aging process. The utility of individual agents in addressing these diseases and conditions is well substantiated; however, the combination of multiple agents, each targeting a particular aspect of oxidative stress, inflammation, and mitochondrial energetic deficiency, is expected to prove more beneficial than the administration of any individual agent alone. The advantage of such a combination therapy is likely derived from the synergy and additive effects of a group of agents wherein each agent may favorably influence or modulate the activities of the others. The cross-talk modulation of a combination of three or four such agents is complex and only beginning to be understood. The specific set of agents disclosed herein is particularly selected to achieve additive and synergistic benefits by balancing the relative types of contributions each can deliver.

Indeed, as shown herein in a reductionist in vitro model in which a human lung epithelial cell line was exposed to severe oxidant and hypoxic stress (by incubating with hydrogen peroxide), whereas addition of NMN alone resulted in a 14% reduction in the elevation in lactate dehydrogenase (LDH) release produced following the exposure to hydrogen peroxide, addition of a combination of NMN, resveratrol and hydroxytyrosol resulted in a 57% reduction in the elevation in LDH release, indicating that while the effect of NMN only is minimal, it is substantially augmented by the concomitant inclusion of resveratrol and hydroxytyrosol.

In another aspect, the present invention provides a drink, beverage, or a dietary supplement, comprising a composition as defined above, e.g., a nutraceutical composition as defined above.

In a further aspect, the present invention relates to a method of anti-aging and anti-inflammatory treatment in a subject in need thereof, said method comprising concomitantly administering to said subject an effective amount of a combination of NMN or an analog thereof; resveratrol; and at least one of ergothioneine or a salt thereof, and hydroxytyrosol. In a particular such aspect, disclosed herein is a method of anti-aging and anti-inflammatory treatment, comprising administering to a subject in need thereof an effective amount of a composition as defined above. The benefits of such an anti-aging and anti-inflammatory treatment include inter alia increasing longevity; boosting energy; decreasing inflammation; improving cognition; and/or decreasing/attenuating deterioration of liver damage, cataract, Alzheimer's disease, diabetes, heart disease, or oxidant mediated diseases, in said subject.

In yet a further aspect, the present invention relates to a composition as defined above, for use in anti-aging and anti-inflammatory treatment, e.g., wherein said composition is formulated as a pharmaceutical composition, or wherein said composition is formulated as a nutraceutical composition and used as a dietary supplement rich in NMN and antioxidants.

### DETAILED DESCRIPTION OF THE INVENTION

In one aspect, disclosed herein is an anti-aging and anti-inflammatory composition comprising NMN or an analog thereof, resveratrol; and at least one of ergothioneine or a salt thereof, and hydroxytyrosol.

NMN (3-carbamoyl-1-[5-O-(hydroxyphosphinato)-β-D-ribofuranosyl] pyridinium; or [(2R,3S,4R,5R)-5-(3-carbamoylpyridin-1-ium-1-yl)-3,4-dihydroxyoxolan-2-yl]methyl hydrogen phosphate) is a nucleotide derived from ribose and nicotinamide, which is naturally found in fruits and vegetables such as edamame, broccoli, cabbage, cucumber, and avocado. Like nicotinamide riboside (NR; 3-carbamoyl-1-[(2R,3R,4S,5R)-3,4-dihydroxy-5-(hydroxymethyl)oxolan-2-yl]pyridin-1-ium), NMN is a derivative of niacin, and humans have enzymes that can use NMN to generate nicotinamide adenine dinucleotide (NAD). Since NADH is a cofactor for processes inside the mitochondria, for sirtuins (a family of signaling proteins involved in metabolic regulation) and for poly (ADP-ribose) polymerase (PARP) (a family of proteins involved in cellular processes such as DNA repair, genomic stability, and programmed cell death), NMN has been studied in animal models as a potential neuroprotective and anti-aging and anti-inflammatory agent, and products containing NMN are currently marketed as dietary supplements. In a clinical trial carried out in 2021. NMN has been found to improve muscular insulin sensitivity in prediabetic women.

The molecular structures of NMN and nicotinamide riboside (NR) are roughly the same, except NMN has an added phosphate group, making it a larger molecule. In fact, some research suggests that when administered exogenously, NMN must be converted to NR in order to enter a cell, where NAD+ biosynthesis occurs, and be re-phosphorylated back to NMN.

In certain embodiments, the anti-aging and anti-inflammatory composition disclosed comprises NMN, and in other embodiments, said composition comprises an NMN analog. In certain particular embodiments, said NMN analog is NAD, which is a coenzyme central to metabolism, found in all living cells and consisting of two nucleotides, one containing adenine nucleobase and the other nicotinamide, linked through their phosphate groups. NAD exists in both oxidized and reduced forms, abbreviated as NAD⁺ and NADH, respectively, and said analog may thus be any one of these forms. In other particular embodiments, said NMN analog is NR, which is a pyridine-nucleoside similar to vitamin B3, functioning as a precursor to NAD+. In further particular embodiments, said NMN analog is niacinamide (known also as nicotinamide, NAM; pyridine-3-carboxamide), which is the active form of vitamin B3 and a component of NAD, currently use as a dietary supplement and medication to prevent and treat pellagra (niacin deficiency). Preferred anti-aging and anti-inflammatory compositions according to the present invention are those comprising NMN.

Resveratrol (3,4′,5-trihydroxy-trans-stilbene; 5-[(E)-2-(4-hydroxyphenyl)ethen-1-yl]benzene-1,3-diol) is a stilbenoid (a stilbene derivative), a type of natural phenol, and a phytoalexin produced by several plants in response to injury or when the plant is under attack by pathogens such as bacteria or fungi. Sources of resveratrol in food include the skin of grapes, blueberries, raspberries, mulberries, and peanuts.

Resveratrol reveals a wide range of biological properties including anti-glycation, antioxidant, anti-inflammation, neuroprotective, anti-cancer, and anti-aging and anti-inflammatory activity in various in vitro and in vivo experimental models. Resveratrol has been reported to extend life span in several different subjects including the fish species Nothobranchius furzeri and Nothobranchius guentheri, as well as Drosophila melanogaster, honeybee and mice. Roggerio et al. (2018) observed that administration of resveratrol (500 mg/day) in healthy and slightly overweight subjects resulted in higher gene expression and serum concentration of sirtuin-1. Sirtuins exhibit a broad spectrum of activity, including anti-ageing and anti-inflammatory effects, inhibition of degenerative disorders such as liver steatosis, as well as improvement of endothelial function, and prevention of cancer. Recent report shows that short-term injection of resveratrol in postovulatory oocyte delayed the aging process of oocytes in middle-aged mice by promoting the expression of sirtuin-1, reducing ROS generation, and ameliorating mitochondrial function (Galiniak et al., 2019).

Ergothioneine (L-ergothioneine; (2S)-3-(2-sulfanylidene-2,3-dihydro-1H-imidazol-4-yl)-2-(trimethylazaniumyl)propanoate) is a naturally occurring amino acid, more specifically a thiourea derivative of histidine, containing a sulfur atom on the imidazole ring. This compound occurs in relatively few organisms (actinomycetota, “cyanobacteria”, and certain fungi), and in humans, it is acquired exclusively through diet, and accumulates in erythrocytes, bone marrow, liver, kidney, seminal fluid, and eyes.

According to the literature, ergothioneine can accumulate at high levels in the body from diet and may play important physiological roles in human health and development, and possibly in prevention and treatment of disease. Yet, blood levels of ergothioneine decline with age and onset of various diseases (Cheah and Halliwell, 2021). As described in this publication, this decline suggests that lower blood levels of ergothioneine are a risk factor for age-related disorders and supplementation could be beneficial. Indeed, Ames (2018) suggested that ergothioneine should be classified as a “longevity vitamin”, i.e., it may play a role in lowering the risk, or progression, of age-related disorders, and promote healthy longevity. Consistent with this, a study investigating 131 blood metabolites and their association with frailty, identified that a declining blood ergothioneine level correlates with markers associated with frailty (Kameda et al., 2020).

Several studies have identified decreased levels of ergothioneine in certain tissues relative to controls, in subjects with various diseases, suggesting potential interventions with administered ergothioneine to raise the levels as both a therapeutic and possibly a preventative agent. Studies in animals and humans have found no toxicity or adverse effects to be associated with ergothioneine administration, even at high doses (Halliwell et al., 2018).

Hydroxytyrosol (4-(2-hydroxyethyl)benzene-1,2-diol) is a phenylethanoid, a type of phenolic phytochemical with antioxidant properties in vitro. In nature, hydroxytyrosol is mainly found in olives, olive leaves, and olive oil in the form of its elenolic acid ester, eleuropein.

Hydroxytyrosol has been hypothesized to exert a wide range of biological effects, cardioprotective, anticancer, neuroprotective antimicrobial, beneficial endocrine and other effects. Although it has been extensively studied, the exact molecular mechanisms underlying many of these actions are yet to be fully clarified. Initially, the wide variety of hydroxytyrosol biological activities was associated with its strong antioxidant activity. Hydroxytyrosol acts as free radical-scavenger and metal-chelator. The high antioxidant efficiency of hydroxytyrosol is attributed to the presence of the o-dihydroxyphenyl moiety. It mainly acts as chain breaker by donating a hydrogen atom to peroxyl-radicals (ROO*). In this way fairly reactive ROO* is replaced with hydroxytyrosol* radical, unreactive due to the presence of intramolecular hydrogen bond in the phenoxy radical. However, it has also been proposed that hydroxytyrosol may confer additional antioxidant protection by increasing the endogenous defense systems against an oxidative stress, by activating different cellular signaling pathways. One of the proposed mechanisms involves the hydroxytyrosol-mediated induction of phase II detoxifying enzymes via nuclear factor E2-related factor 2 (Nrf2) activation in different tissues. Also, the interaction of hydroxytyrosol with microRNAs (miRNAs) should be a potential molecular target for eliciting its biological effects. miRNAs exert important regulatory actions on gene expression not only under physiological circumstances but also in disease. Indeed, miRNAs often target multiple functionally related genes and extensively interfere with biological processes, rendering them good candidates for therapeutic and/or nutraceutical interventions. Hydroxytyrosol modulates the expression of several miRNAs. In addition, hydroxytyrosol possesses significant anti-inflammatory activity. In vitro and in vivo evidence show the attenuation of pro-inflammatory agents inducible nitric oxide synthase (iNOS), cyclooxygenase-2 (COX-2), tumor necrosis factor-α (TNF-α) and interleukin (IL)-1β expression, inhibition of granulocytes and monocytes activation by hydroxytyrosol (Markovic et al., 2019; D'Angelo et al., 2020; Serreli and Deiana, 2020).

Based on the aforesaid, it is expected that the anti-aging and anti-inflammatory composition of the present invention, comprising NMN, or an analog thereof; resveratrol; and at least one ergothioneine or a salt thereof and hydroxytyrosol, will be highly beneficial in conditions of aging and inflammation. NMN and its analogs are expected to restore energetic deficiency at the mitochondrial level. Resveratrol, hydroxytyrosol, and ergothioneine are expected to counteract inflammatory signaling pathways and to provide direct and indirect antioxidant activities across a broad range of diverse oxidative and nitrosative processes.

According to the present invention, the anti-aging and anti-inflammatory composition disclosed may comprise ergothioneine or a salt thereof. Such a salt may be a pharmaceutically acceptable salt (in case the composition disclosed is a pharmaceutical composition) or a salt acceptable for food and drink (in case said composition is a nutraceutical composition), and may be either an acidic or basic salt. Suitable acidic pharmaceutically acceptable salts include, without being limited to, the mesylate salt, the maleate salt, the fumarate salt, the tartrate salt, the hydrochloride salt, the hydrobromide salt, the esylate salt, the p-toluenesulfonate salt, the benzenesulfonate salt, the benzoate salt, the acetate salt, the phosphate salt, the sulfate salt, the citrate salt, the carbonate salt, and the succinate salt. Basic pharmaceutically acceptable salts include, without limiting, metal salts such as alkali metal salts, e.g., lithium, sodium and potassium salts, and alkaline earth metal salts, e.g., calcium and magnesium salts; as well as salts of ammonium (NH₄⁺) or an organic cation derived from an amine of the formula R₄N⁺, wherein each one of the Rs independently is selected from H, C₁-C₂₂, preferably C₁-C₆ alkyl, such as methyl, ethyl, propyl, isopropyl, n-butyl, sec-butyl, isobutyl, tert-butyl, n-pentyl, 2,2-dimethylpropyl, n-hexyl, and the like, phenyl, or heteroaryl such as pyridyl, imidazolyl, pyrimidinyl, and the like, or two of the Rs together with the nitrogen atom to which they are attached form a 3-7 membered ring optionally containing a further heteroatom selected from N, S and O, such as pyrrolydine, piperidine and morpholine.

In certain embodiments, the anti-aging and anti-inflammatory composition disclosed comprises NMN or an analog thereof as defined hereinabove, preferably NMN; resveratrol; and ergothioneine or salt thereof.

In other embodiments, the anti-aging and anti-inflammatory composition disclosed comprises NMN or an analog thereof as defined hereinabove, preferably NMN; resveratrol; and hydroxytyrosol.

In further embodiments, the anti-aging and anti-inflammatory composition disclosed comprises NMN or an analog thereof as defined hereinabove, preferably NMN; resveratrol; ergothioneine or a salt thereof; and hydroxytyrosol.

In certain embodiments, disclosed herein is an anti-aging and anti-inflammatory composition according to any one of the embodiments above, wherein the amount of said NMN or analog thereof in said composition is from about 101 mg to about 1500 mg, e.g., from about 150 mg to about 1400 mg, from about 200 mg to about 1300 mg, from about 250 mg to about 1200 mg, from about 300 mg to about 1100 mg, from about 350 mg to about 1000 mg, from about 400 mg to about 900 mg, from 450 mg to 800 mg, or from 500 mg to 700 mg; the amount of said resveratrol in said composition is from about 50 mg to about 500 mg, e.g., from about 75 mg to about 450 mg, from about 100 mg to about 400 mg, from about 125 mg to about 350 mg, from about 150 mg to about 300 mg, or from about 175 mg to about 250 mg; the amount of said ergothioneine or salt thereof, when present, in said composition is from about 3 mg to about 25 mg (more specifically about 3-10 mg or about 10-25 mg), e.g., from about 3 mg to about 10 mg, from about 10 mg to about 25 mg, from about 4 mg to about 22.5 mg, from about 5 mg to about 20 mg, from about 6 mg to about 17.5 mg, from about 7 mg to about 15 mg, from about 8 mg to about 12.5 mg, or from about 9 mg to about 10 mg; and the amount of said hydroxytyrosol, when present, in said composition is from about 20 mg to about 100 mg, e.g., from about 25 mg to about 90 mg, from about 30 mg to about 90 mg, from about 35 mg to about 80 mg, from about 40 mg to about 70 mg, or from about 45 mg to about 60 mg.

Maltodextrin (CAS Reg. No. 9050-36-6) is a polysaccharide (more specifically oligosaccharide) consisting of D-glucose units linked with α(1→4) glycosidic bonds in chains of variable length (typically composed of a mixture of chains that vary from 3-17 glucose units long), which is produced from vegetable starch by partial hydrolysis. This polysaccharide may be either moderately sweet or almost flavorless (depending on the degree of polymerization), is extremely water-soluble and easily digestible being absorbed as rapidly as glucose, and may be found as an ingredient in a variety of processed foods. The Food Chemicals Codex lists maltodextrin as a stabilizer, thickener, anticaking agent and bulking agent.

In certain embodiments, disclosed herein is an anti-aging and anti-inflammatory composition according to any one of the embodiments above, wherein said ergothioneine or salt thereof is present, and said composition further comprises maltodextrin stabilizing said ergothioneine. In particular such compositions, the ratio between said ergothioneine or salt thereof and said maltodextrin is from about 1:5 to about 1:50, e.g., about 1:6, 1:7, 1:8, 1:9, 1:10, 1:12, 1:14, 1:16, 1:18, 1:20, 1:25, 1:30, 1:35, 1:40, or 1:45, by weight, respectively.

The anti-aging and anti-inflammatory compositions disclosed herein, according to any one of the embodiments above, may be formulated as pharmaceutical- or nutraceutical compositions optionally further comprising one or more suitable carriers and/or excipients.

In certain embodiments, the composition disclosed is a pharmaceutical composition optionally further comprising a pharmaceutically acceptable carrier and/or excipient.

The term “pharmaceutically acceptable carrier” or “pharmaceutically acceptable excipient” as used herein interchangeably refers to any non-active ingredient such as a solvent, dispersion medium, preservative, antioxidant, coating, isotonic and absorption delaying agent, and the like, that is compatible with pharmaceutical administration, and does not produce an adverse, allergic, or other untoward reaction when administered to a mammal or human as appropriate.

For human administration, compositions should meet sterility, pyrogenicity, and general safety and purity standards as required by, e.g., the U.S. Food and Drug Administration (FDA), or the European Medicines Agency (EMA).

Pharmaceutical compositions as disclosed herein may be prepared by any conventional technique known in the art, e.g., by uniformly and intimately bringing the active agents i.e., said NMN or analog thereof, said resveratrol, and one or both of said ergothioneine or salt thereof and said hydroxytyrosol, into association with a liquid carrier, a finely divided solid carrier, or both, and then, if necessary, shaping the product into the desired formulation.

The pharmaceutical composition of the invention may be formulated for any suitable administration route, but they are preferably formulated for oral, sublingual, sublabial, buccal, rectal, vaginal, subcutaneous, topical, or ophthalmic administration.

Pharmaceutical compositions formulated for oral administration may be in the form of a liquid, e.g., a solution in an edible solvent such as ethanol, tincture, syrup, or elixir; a semi-solid; or a solid such as tablets, caplets, pills, troches, lozenges, dispersible powder or granules, hard or soft capsules, and sachets.

Useful dosage forms of the pharmaceutical composition include orally disintegrating systems including, but not limited to, solid, semi-solid and liquid systems including disintegrating or dissolving tablets, soft or hard capsules, gels, fast dispersing dosage forms, controlled dispersing dosage forms, caplets, films, wafers, ovules, granules, buccal/mucoadhesive patches, powders, freeze dried (lyophilized) wafers, chewable tablets which disintegrate with saliva in the buccal/mouth cavity and combinations thereof. Useful films include, but are not limited to, single layer stand-alone films and dry multiple layer stand-alone films.

In certain embodiments, the pharmaceutical compositions are formulated for oral administration, and are in the form of a matrix tablet, wherein the release of the active agents is controlled by having said active agents diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other embodiments, the tablets are formulated as bi- or multi-layer tablets, made up of two or more distinct layers of granulation compressed together with the individual layers lying one on top of another, with each separate layer containing a different active agent. Bilayer tablets have the appearance of a sandwich since the edge of each layer or zone is exposed.

Pharmaceutical compositions for oral administration might be formulated so as to inhibit the release of one or more of the active agents in the stomach, i.e., delay the release of said active agent(s) until at least a portion of the dosage form has traversed the stomach, in order to avoid the acidity of the gastric contents from hydrolyzing the active agent. Particular such compositions are those wherein the active agent is coated by a pH-dependent enteric-coating polymer. Examples of pH-dependent enteric-coating polymer include, without being limited to, Eudragit® S (poly(methacrylicacid, methylmethacrylate), 1:2), Eudragit® L 55 (poly (methacrylicacid, ethylacrylate), 1:1), Kollicoat® (poly(methacrylicacid, ethylacrylate), 1:1), hydroxypropyl methylcellulose phthalate (HPMCP), alginates, carboxymethylcellulose, and combinations thereof. The pH-dependent enteric-coating polymer may be present in the composition in an amount from about 10% to about 95% by weight of the entire composition.

Another contemplated formulation is depot systems, based on biodegradable polymers. As the polymer degrades, the active agent(s) is slowly released. The most common class of biodegradable polymers is the hydrolytically labile polyesters prepared from lactic acid, glycolic acid, or combinations of these two molecules. Polymers prepared from these individual monomers include poly (D,L-lactide) (PLA), poly (glycolide) (PGA), and the copolymer poly (D,L-lactide-co-glycolide) (PLG).

Pharmaceutical compositions for oral administration may further comprise one or more agents selected from sweetening agents, flavoring agents, coloring agents and preserving agents in order to provide pharmaceutically elegant and palatable preparations. In addition, said compositions may comprise one or more pharmaceutically acceptable excipients. For example, a tablet may comprise at least one filler, e.g., lactose, ethylcellulose, microcrystalline cellulose, silicified microcrystalline cellulose; at least one disintegrant, e.g., cross-linked polyvinylpyrrolidinone; at least one binder, e.g., polyvinylpyridone, hydroxypropylmethyl cellulose; at least one surfactant, e.g., sodium laurylsulfate; at least one glidant, e.g., colloidal silicon dioxide; and at least one lubricant, e.g., magnesium stearate.

Pharmaceutical compositions formulated for parenteral administration, i.e., for administration elsewhere in the body than the mouth and alimentary canal, may be in the form of a sterile, optionally injectable, aqueous or oleaginous suspension, which may be formulated according to the known art using suitable dispersing, wetting or suspending agents. The sterile injectable preparation may also be an injectable solution or suspension in a non-toxic parenterally acceptable diluent or solvent. Acceptable vehicles and solvents that may be employed include, without limiting, water, Ringer's solution, polyethylene glycol (PEG), 2-hydroxypropyl-β-cyclodextrin (HPCD), a surfactant such as Tween-80, and isotonic sodium chloride solution.

In certain embodiments, the composition disclosed is a nutraceutical composition optionally further comprising a nutraceutically acceptable carrier and/or excipient, formulated, e.g., for oral, sublingual, buccal, or sublabial administration.

In certain embodiments, a nutraceutical composition as referred to herein further comprises a binder. The term “binder” or “binding agent” used herein interchangeably refers to any material or substance that holds or draws other materials together to form a mechanically and/or chemically cohesive whole, by adhesion or cohesion. More specifically, binders are liquid or dough-like substances that harden by a chemical or physical process and bind fibers, filler powder and other particles added into it. Examples of binding agents include, without being limited to, a saccharide such as sucrose and glucose (e.g., liquid glucose); a polysaccharide such as starch (e.g., starch paste and pregelatinized starch) and cellulose; a natural binder such as acacia (also known as gum Arabic), tragacanth, gelatin, and alginic acid; or a synthetic or semi-synthetic polymer such as a cellulose-based polymer (e.g., methylcellulose, ethylcellulose, hydroxypropyl cellulose, hydroxypropyl methylcellulose (HPMC), and sodium carboxymethyl cellulose), polyvinyl pyrrolidone (PVP), polyvinyl alcohol, PEG, and a polymethacrylate (e.g., poly(methyl methacrylate) (PMMA)).

Nutraceutical compositions as referred to herein may be in the form of a liquid, e.g., a solution in an edible solvent such as ethanol, tincture, syrup, or elixir; a semi-solid; or a solid such as tablets, caplets, pills, troches, lozenges, dispersible powder or granules, hard or soft capsules, and sachets.

In another aspect, disclosed herein is a drink, beverage, or food supplement, comprising an anti-aging and anti-inflammatory composition according to any one of the embodiments above, i.e., a composition comprising NMN or an analog thereof, preferably NMN; resveratrol; and at least one of ergothioneine or a salt thereof, and hydroxytyrosol.

In a further aspect, disclosed herein is a method of anti-aging and anti-inflammatory treatment in a subject in need thereof, said method comprising concomitantly administering to said subject an effective amount of a combination of NMN or an analog thereof, preferably NMN; resveratrol; and at least one of ergothioneine or a salt thereof, and hydroxytyrosol. In a particular such aspect, the present invention relates to a method of anti-aging and anti-inflammatory treatment in a subject in need thereof, said method comprising administering to said subject an effective amount of an anti-aging and anti-inflammatory composition as defined in any one of the embodiments above, i.e., a composition comprising NMN or an analog thereof, preferably NMN; resveratrol; and at least one of ergothioneine or a salt thereof, and hydroxytyrosol.

The term “subject” as used herein refers to any mammal, e.g., a human, non-human primate, horse, ferret, dog, cat, cow, and goat. In a preferred embodiment, the term “subject” denotes a human, i.e., an individual.

The phrase “anti-aging and anti-inflammatory treatment” as used herein refers to administration of an amount of the drug (active agent) combination disclosed, i.e., NMN or an analog thereof; resveratrol; and at least one of ergothioneine (or a salt thereof) and hydroxytyrosol, which is effective when given either pharmaceutically or nutraceutically in subjects who suffer from inflammatory disease and those who wish to slow the natural process of aging.

The term “therapeutically effective amount” as used herein means an amount or dose of the drug combination disclosed, when formulated as a pharmaceutical composition, that is useful to alleviate inflammatory disease and slow the natural process of aging. The amount must be effective to achieve the desired therapeutic effect as described above, depending, inter alia, on the severity of the condition to be treated and the treatment regime. The therapeutically effective amount is typically determined in appropriately designed clinical trials (dose range studies), and the person versed in the art will know how to properly conduct such trials to determine said amount, i.e., the dose of said drug combination that is therapeutically effective. The therapeutically effective amount of the drug combination administered according to the method of the invention depends on both the effective amount of each one of drugs included within the combination and the therapeutic effect achieved when these three or four drugs are administered as a combination.

In certain embodiments, the method of the present invention is specifically aimed at increasing longevity, boosting energy, decreasing inflammation, improving cognition, or decreasing/attenuating deterioration of liver damage, cataract, Alzheimer's disease, diabetes, heart disease, or oxidant mediated diseases, in said subject.

In yet a further aspect, disclosed herein is an anti-aging and anti-inflammatory composition as defined in any one of the embodiments above, i.e., a composition comprising NMN or an analog thereof, preferably NMN; resveratrol; and at least one of ergothioneine or a salt thereof, and hydroxytyrosol, for use in anti-aging and anti-inflammatory treatment. In certain embodiments, said composition is formulated as a pharmaceutical composition, and in other embodiments, said composition is formulated as a nutraceutical composition and used as a dietary supplement rich in NMN and antioxidants.

In certain embodiments, the use of said anti-aging and anti-inflammatory composition according to the present invention is aimed at increasing longevity, boosting energy, decreasing inflammation, improving cognition, or decreasing/attenuating deterioration of liver damage, cataract, Alzheimer's disease, diabetes, heart disease, or oxidant mediated diseases.

The invention will now be illustrated by the following non-limiting Examples.

### EXAMPLES

### Example 1. A Combination of NMN, Resveratrol, and Hydroxytyrosol Reduces the Elevation in LDH Release Following a Cellular Injury

In order to model cytoprotective effects of the invention, a reductionist in vitro model was employed wherein a human lung epithelial cell line (A549) was exposed to severe oxidant and hypoxic stress. Cultured A549 cells were placed in 96 well plates, incubated in Dulbecco's Modified Eagle's Medium (DMEM), and allowed to grow to confluence over 72 hours. Cellular injury was created by the addition into the media of 100 micromolar hydrogen peroxide (H₂O₂), followed by incubation at 37° C. for 18 hours in an atmosphere of 95% nitrogen and 5% carbon dioxide, which maintained a pH in the incubation media of 7.4.

Three experimental arms were evaluated, using quadruplicate replicates, as follows: a) media control, b) NMN, and c) NMN plus resveratrol and hydroxytyrosol. A control arm was examined that was not exposed to hydrogen peroxide. The treatment agents were added such that their final concentration in the media was 10 micromolar. lactate dehydrogenase (LDH) was assayed in the media after 18 hours using a commercial kit, based upon a colorimetric change quantified by visible spectrophotometry.

Addition of NMN alone resulted in a 14% reduction in the elevation in LDH release produced by addition of hydrogen peroxide alone. In contrast, the use of a combination of NMN, resveratrol, and hydroxytyrosol resulted in a 57% reduction in the elevation in LDH release produced by addition of hydrogen peroxide alone. These results indicated that NMN is minimally effective when given alone, but its effect is augmented substantially by the concomitant inclusion of resveratrol and hydroxytyrosol.

### REFERENCES

Ames, B.N., Prolonging healthy aging: longevity vitamins and proteins. Proc. Natl. Acad. Sci. U.S.A. 2018, 115(43), 10836-10844
Cheah, I.K.; Halliwell, B., Ergothioneine, recent developments. Redox Biol., 2021, 42, 101868
D'Angelo, C.; Franceschelli, S.; Quiles, J.L.; Speranza, L., Wide biological role of hydroxytyrosol: possible therapeutic and preventive properties in cardiovascular diseases. Cells, 2020, 9(9), 1932
Galiniak, S.; Aebisher, D.; Bartusik-Aebisher, D., Health benefits of resveratrol administration. Acta Biochimica Polonica, 2019, 66(1), 13-21
Halliwell, B.; Cheah, I.K.; Tang, R.M.Y., Ergothioneine—a diet-derived antioxidant with therapeutic potential. FEBS Letters, 2018, 592(20), 3357-3366
Kameda M., Teruya T., Yanagida M., Kondoh H. Frailty markers comprise blood metabolites involved in antioxidation, cognition, and mobility. Proc. Natl. Acad. Sci. U.S.A. 2020, 117(17), 9483-9489
Markovic, A.K.; Toric, J.; Barbaric, M., Brala, C.J., Hydroxytyrosol, tyrosol and derivatives and their potential effects on human health. Molecules, 2019, 24(10), 2001
Roggerio, A.; Strunz, C.M.C.; Pacanaro, A.P.; Leal, D.P.; Takada, J.Y.; Avakian, S.D.; Mansur, A.dP., Gene expression of sirtuin-1 and endogenous secretory receptor for advanced glycation end products in healthy and slightly overweight subjects after caloric restriction and resveratrol administration. Nutrients, 2018, 10(7), 937
Serreli, G.; Deiana, M., Extra virgin olive oil polyphenols: modulation of cellular pathways related to oxidant species and inflammation in aging. Cells, 2020, 9(2), 478

1. An anti-aging and anti-inflammatory composition comprising nicotinamide mononucleotide (NMN) or nicotinamide riboside (NR); resveratrol; and at least one of ergothioneine or a salt thereof, and hydroxytyrosol.

2. (canceled)

3. The composition of claim 1, comprising said NMN or NR; said resveratrol; and said ergothioneine or a salt thereof.

4. The composition of claim 1, comprising said NMN or NR; said resveratrol; and said hydroxytyrosol.

5. The composition of claim 1, comprising said NMN or NR; said resveratrol; said ergothioneine or a salt thereof; and said hydroxytyrosol.

6. The composition of claim 1, wherein the amount of said NMN or NR in said composition is from about 101 mg to about 1500 mg; the amount of said resveratrol in said composition is from about 50 mg to about 500 mg; the amount of said ergothioneine or salt thereof, when present, in said composition is from about 3 mg to about 25 mg; and the amount of said hydroxytyrosol, when present, in said composition is from about 20 mg to about 100 mg.

7. The composition of claim 1, wherein said ergothioneine or salt thereof is present, and said composition further comprises maltodextrin.

8. The composition of claim 7, wherein the ratio between said ergothioneine or salt thereof and said maltodextrin in said composition is from about 1:5 to about 1:50 by weight, respectively.

9. The composition of claim 1, formulated as a pharmaceutical composition further comprising a pharmaceutically acceptable carrier.

10. The composition of claim 9, for oral, sublingual, rectal, vaginal, subcutaneous, topical, or ophthalmic administration.

11. The composition of claim 1, formulated as a nutraceutical composition further comprising a nutraceutically acceptable carrier.

12. The composition of claim 11, in the form of a tablet, caplet, pill, hard or soft capsule, troche, lozenge, or dispersible powder or granules.

13. A drink, beverage, or a dietary supplement, comprising the composition according to claim 1.

14. A method of anti-aging and anti-inflammatory treatment in a subject in need thereof, said method comprising administering to said subject an effective amount of the composition according to claim 1.

15. The method of claim 14, for increasing longevity; boosting energy; decreasing inflammation; improving cognition; or decreasing/attenuating deterioration of liver damage, cataract, Alzheimer's disease, diabetes, heart disease, or oxidant mediated diseases, in said subject.

16-18. (canceled)

19. The composition of claim 8, wherein the ratio between said ergothioneine or salt thereof and said maltodextrin in said composition is about 1:20 by weight, respectively.

20. The composition of claim 11, in the form of a liquid, semi-solid, or solid.
